(19) United States
(12) Patent Application Publication
(10) Pub. No.: US 2004/0034101 A1
(43) Pub. Date: Feb. 19, 2004

(54) Treatment and prevention of depression secondary to pain (DSP)

(75) Inventors: Srinivas G. Rao, San Diego, CA (US); Jay D. Kranzler, LaJolla, CA (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(73) Assignee: Cypress Bioscience, Inc.

(21) Appl. No.: 10/628,141

(22) Filed: Jul. 24, 2003

Related U.S. Application Data
(63) Continuation-in-part of application No. 10/028,547, filed on Dec. 19, 2001, now Pat. No. 6,602,911.
Application No. 10/028,547 is a continuation-in-part of application No. 10/014,149, filed on Nov. 5, 2001, now Pat. No. 6,635,675.
(60) Provisional application No. 60/443,035, filed on Jan. 28, 2003.
(60) Provisional application No. 60/398,676, filed on Jul. 24, 2002.

(51) Int. Cl. A61K 31/165

(52) U.S. Cl. 514/619

(57) ABSTRACT

Methods for the prevention or treatment of a typical depression secondary to pain (DSP) have been developed. The method generally involves administering an effective amount of a monoamine re uptake inhibitor to treat or prevent symptoms of DSP. In a preferred embodiment, a therapeutically effective amount of a dual serotonin norepinephrine reuptake inhibitor (SRNI) compound of a specific type, or a pharmaceutically acceptable salt thereof is administered. The most preferred SNRI compounds are non-tricyclic SNRIs, wherein serotonin reuptake inhibition is greater than norepinephrine reuptake inhibition; and NSRIs, wherein norepinephrine reuptake inhibition is greater than serotonin reuptake inhibition. The most preferred compound is milnacipran or a bioequivalent or pharmaceutically acceptable salt thereof. Other preferred compounds are duloxetine and venlafaxine or a bioequivalent or pharmaceutically acceptable salt thereof. In yet another embodiment, a therapeutically effective amount of a non-tricyclic triple reuptake inhibitor (“TRI”) compound of a specific type, or a pharmaceutically acceptable salt thereof, is administered. The TRI compounds are characterized by their ability to block the reuptake (and, hence, increase central concentrations of) the three primary brain monoamines: serotonin, noradrenaline, and dopamine.

This application is a continuation-in-part of U.S. Ser. No. 10/028,547 entitled “Method of Treating Fibromyalgia” filed Dec. 19, 2001, which is a continuation-in-part of U.S. Ser. No. 10/014,149 entitled “Method of Treating Chronic Fatigue Syndrome” filed Nov. 5, 2001, and also claims priority to U.S. S. No. 60/398,676 entitled “Treatment of Pain-Associated Depression (PAD)” filed Jul. 24, 2002 and to U.S. S. No. 60/443,035 entitled “Treatment of Pain-Associated Depression (PAD)” filed Jan. 28, 2003.

### FIELD OF THE INVENTION

The present invention is in the field of treating a typical depression associated with a chronic pain state.

### BACKGROUND OF THE INVENTION

Chronic pain is thought to result in a high incidence of clinical depression with some estimates suggesting that almost two thirds of patients with chronic non-malignant pain have coexisting symptoms of depression or anxiety. One problem in treating chronic pain is that little is known about what causes the pain state. Acute pain occurs when an individual experiences an acute injury. Chronic pain is more complex because it often occurs in the absence of any ongoing illness or disease and is often intractable using conventional analgesics. Chronic pain usually occurs following an acute injury, but continues for an unknown reason after the injured area has healed. Chronic pain can also be caused by an ongoing condition like Chronic Fatigue Syndrome (CFS), Fibromyalgia syndrome (FMS), arthritis, or an illness like cancer or multiple sclerosis. The cause of pain cannot be removed or treated and the pain itself cannot be relieved. This gives rise to feelings of helplessness, decreased energy, low self-esteem and social support (Brown et al Br J Psychiatry 147, 612-22 (1985)). This pattern often leads to depression, anxiety and frustration which further exacerbate the pain. Chronic or intractable pain is often endured over many years or decades. Patients suffering from chronic pain often develop emotional problems which can lead to depression and in worst cases, attempted suicide.

High degrees of co-morbidity between chronic pain and depression have been reported in the literature, with approximately 50% of chronic pain patients displaying significant levels of depression (Romano and Turner Psychol. Bull., 97:18-34 (1985)). Depression is often co-morbid in patients suffering from CFS and FMS. Several studies have reported that depression is also an important predictor of disability in chronic pain patients (e.g., Haley et al. Pain, 23:337-343 (1985); Dworkin et al., Pain, 24:343-353 (1986); Doan, B. and Wadden Pain, 36:75-84 (1989)) as well as a predictor of motivation for treatment (Kerns and Haythornthwaite J. Consult. Clin. Psychol., 56:870-876 (1988)).

Depression refers to an abnormal mood or a collection of symptoms that constitute a psychiatric disorder. Symptoms of depression include disturbances in mood and affect (depressed mood, diminished interest and pleasure in activities), bodily function (weight and appetite changes, psychomotor disturbances, sleep disturbances, fatigue and loss of energy), and cognitive processes (feelings of worthlessness and guilt, concentration difficulties, indecisiveness, thoughts of death or suicide and possibly delusions/hallucinations). These symptoms vary in intensity, duration and frequency and permit classification of depression into different classes. Other symptoms of major depressive episodes include crying spells, self-pity, hopelessness, irritability, brooding, diminished self-esteem, decreased libido, nihilism, social withdrawal, memory impairment, feelings of inadequacy and pessimism. These symptoms are summarized in the American Psychiatric Association's Diagnostic and Statistical Manual of Mental Disorders, 4th Edition, Text Revision (DSM-IV-TR; 1994).

A typical depression is one type of depressive order included in DSM-IV-TR at page 420 about which there has been substantial clinical and research interest. Although at the present time it is not clear how common this diagnosis is in chronic pain patients, there are certainly pain patients expressing the characteristics of a typical depression.

There are at least two broad types of a typical depression that differ from classically defined depression (Davidson et al. Arch. Gen. Psychiatry, 39, 527-34 (1982); Paykel et al. Psychol. Med., 13,:131-9 (1983); Paykel et al, Arch. Gen. Psychiatry, 39:1041-9 (1982)). One is composed of those depressions accompanied by severe anxiety, and also by phobic symptoms, tension, and pain. The other type of a typical depression is characterized by reversed vegetative symptoms, e.g., increased (rather than decreased) appetite, weight, and sleep.

Monoamine neurotransmitters have been implicated in the body's response to both pain and depression. Norepinephrine (NE) and Serotonin (5-HT) are monoamine neurotransmitters originating in the brain and projecting diffusely throughout the central nervous system. 5-HT and NE are involved in modulating pain transmission from the spinal cord to the brain and also governing the body's moods and responses to stress. Electrical stimulation of these brain regions releases 5-HT and NE and has been reported to produce a deep analgesia in both animals and humans (Akil and Liebeskind Brain Res 94:279-296 (1975)). Conversely, depletion of serotonin in the rat results in an enhanced response to pain (Berge et al Brain Res 271:51-64 (1983)). There also appears to be synergistic actions between NE and 5-HT in modulating pain sensation. Studies in the rat show that the analgesia from exogenously administered 5-HT can be blocked by depleting NE in the spinal cord. (Post et al Brain Res 363:18-27 (1986)).

Considerable evidence suggests that depression is due to a decrease in monoamine levels in the central nervous system. Analysis of cerebrospinal fluid in patients with depression has shown decreased levels of serotonin and norepinephrine as well as their respective metabolites. Patients with depression have displayed other indicators of monoamine dysfunction such as decreased serotonin transporter binding, decreased serotonin uptake by platelets and brain tissue, and alterations in peripheral norepinephrine receptors and neuroendocrine responses to norepinephrine. (Owens and Nemeroff Clin Chem 40:288-95 (1995); Delgado et al Arch Gen Psychiatry 47:411-418 (1990), Vetulani and Sulser Nature 257:495-6 (1975); Vetulani et al Naunyn-Schmiedeberg Arch Pharmacol 293:109-114 (1976); Potter and Manji Clin Chemistry 40:279-287 (1994)).

Current treatments for depression increase serotonin via a number of different neurochemical mechanisms. Tricylic antidepressants are a common class of antidepressant that increase concentrations of NE and 5-HT in the synaptic cleft by blocking reuptake or by inhibiting their metabolism. The increased synaptic availability of NE leads to a delayed desensitization of the β-Norepinephrine-receptor-coupled adenylate cyclase system. This biochemical action is shared by most clinically effective antidepressant treatments including electro convulsive therapy (Baker and Greenshaw, Cell Molec. Neurobiol., 9:1-44 (1989)). Drugs that selectively inhibit the re-uptake of 5-HT are effective treatments for depression. Drugs that block re-uptake inhibitors produce a relapse in depression supporting the importance of this class of drug. (Owens and Nemeroff Clin Chem 40:288-95 (1995); Blier and deMontigny Trends Pharmacol Sci 15:220-226 (1994); Delgado et al Arch Gen Psychiatry 47:411-418 (1990). Current treatments for depression include tricyclic antidepressants, monoamine oxidase inhibitors, lithium, selective 5-HT reuptake inhibitors, dual reuptake inhibitors and selective NE reuptake inhibitors. Tricyclic antidepressants and selective 5-HT reuptake inhibitors are generally ineffective in treating symptoms of a typical depression such as pain and anxiety and are generally not considered first line therapies (Joyce et al N Z J Psychiatry 36:384-391 (2002); Stewart et al Neuropsychopharmacology 26:237-245 (2002)). Although monoamine oxidase inhibitors are effective in treating a typical depression, their side effects and prescription-associated dietary restrictions also reduce their suitability as a first-line treatment.

Different types of depression can be characterized by the type, intensity and frequency of symptoms, each responding preferentially to different therapeutic drugs. The diversity in the class of monoamine re-uptake inhibitors is ideal for treating this broad class of mental illness. Different therapeutic compounds with different binding affinities for each monoamine transporter are ideally suited for therapeutic use and offer the subtle differences that are necessary to treat the numerous types of depression.

It is therefore an object of the present invention to provide a method to treat and/or prevent a typical depression which is secondary to pain (DSP).

It is a further object of this invention to provide a method to treat or prevent the a typical depressive component of depression secondary to pain (DSP) as well as the pain (e.g. chronic pain or neuropathic pain).

It is a further object of this invention to provide a method to treat and/or prevent depression secondary to pain (DSP) that will not substantially increase the risk of seizures.

### SUMMARY OF THE INVENTION

Methods for the prevention or treatment of a type of a typical depression secondary to pain (DSP) have been developed. The method generally involves administering an effective amount of a monoamine reuptake inhibitor to treat or prevent DSP. In a preferred embodiment, a therapeutically effective amount of a dual serotonin norepinephrine reuptake inhibitor (SNRI), or a pharmaceutically acceptable salt thereof, is administered. The most preferred SNRI compounds are non-tricyclic SNRIs, wherein serotonin reuptake inhibition is greater than norepinephrine reuptake inhibition; and dual norepinephrine serotonin reuptake inhibitors (NSRIs), wherein norepinephrine reuptake inhibition is greater than serotonin reuptake inhibition. The most preferred compound is milnacipran or a bioequivalent or pharmaceutically acceptable salt thereof. Other preferred compounds are duloxetine, venlfaxine and sibutramine, metabolites, derivatives or a bioequivalent or pharmaceutically acceptable salt of these compounds. In another embodiment, a therapeutically effective amount of a non-tricyclic triple reuptake inhibitor (“TRI”), or a pharmaceutically acceptable salt thereof, is administered. The TRI compounds are characterized by their ability to block the reuptake (and, hence, increase central concentrations of) the three primary brain monoamines: serotonin, noradrenaline, and dopamine.

The compound is administered in an effective amount to treat symptoms of a typical depression secondary to pain such as anxiety, pain and neurovegetative symptoms.

### BRIEF DESCRIPTION OF THE DRAWINGS

FIG. 1 is a bar graph showing the effects of a twice a day (BID) or once a day (QD) adminstration of milnacipran on FMS patient global condition scores. Patients were asked to rate their condition at the end of the treatment on a seven point scale with 1 being worse and 7 being greatly improved. Placebo group scores are also shown (PL).

FIG. 2 is a bar graph showing scores for test FMS subjects using the Beck Depression Index. Scores were taken before the study to establish baseline and at the endpoint of the study to determine change due to treatment. Milnacipran treated subjects once-a-day (QD) or twice-a-day (BID) reported significant improvements over placebo group.

FIG. 3 is a bar graph showing ITT Response Rate in a binary responder analysis of pain. Scores were derived from data acquired for two weeks before treatment of FMS patients to establish a baseline and for two weeks at the end. Both milnacipran-treated groups demonstrated improved ITT Response Rates over placebo.

### DETAILED DESCRIPTION OF THE INVENTION

Abbreviations

| | |
|---|---|
| DSP | Depression Secondary to Pain |
| CFS | Chronic Fatigue Syndrome |
| FMS | Fibromyalgia Syndrome |
| 5-HT | serotonin |
| NE | norepinephrine (noradrenaline) |
| DA | dopamine |
| NMDA | N-methyl D-aspartate |
| NSAIDs | non-steroidal anti-inflammatory drugs |
| SSRIs | selective serotonin reuptake inhibitors |
| TCAs | tricyclic antidepressants |
| SNRIs | dual serotonin norepinephrine reuptake inhibitors, where |
| | serotonin reuptake exceeds norepinephrine reuptake. |
| NSRI | dual norepinephrine reuptake inhibitor |
| | where norepinephrine reuptake exceeds serotonin |
| | reuptake. |
| TRI | a compound that blocks the reuptake of 5-HT, NE, and |
| | DA |

I. Disorders to be Treated

Depressive disorders are widely prevalent psychiatric conditions characterized by the presence of negative affect, with or without mania (e.g., bipolar disorder). A number of sub-types have been described, based upon the presence, chronicity, and severity of specific symptomatology which can determine a patient's treatment. The symptoms of depressive disorders are numerous, and vary in intensity, duration, and frequency. A diagnosis of a depressive disorder can be made when a number of these symptoms have been present for a given period of time. These symptoms are listed in the American Psychiatric Association's Diagnostic and Statistical Manual of Mental Disorders, fourth Edition, Text Revision (DSM-IV-TR; 1994). The symptoms specified by these criteria, reflect marked alterations in mood and affect (depressed mood, diminished interest and pleasure in activities), bodily functioning (weight and appetite changes, sleep disturbances, psychomotor disturbances, fatigue and loss of energy), and cognitive processes (feelings of worthlessness and guilt, concentration difficulties and indecisiveness, thoughts of death and suicide, and, may include delusions and hallucinations).

Symptoms of depressive disorders that are not listed in these DSM-IV-TR criteria, but that are often part of a major depressive episode include crying spells, self-pity, hopelessness, irritability, brooding, diminished self-esteem, loss of libido, nihilistic ideas, social withdrawal, memory impairment, feelings of inadequacy, and pessimism (Beck A. T., Depression: clinical, experimental, and theoretical aspects. New York: Hoeber (1967)).

A typical depression is one type of depressive disorder. Some of these patients fulfill DSM-IV-TR criteria for major depression or dysnthymia but manifest their depression with symptoms that are considered a typical; others do not satisfy DSM-IV-TR criteria for a specific mood disorder but appear to be suffering from an a typical depression.

A typical depression is a depressed affect, with the ability to feel better temporarily in response to positive life effect (mood reactivity), plus two or more neurovegetative symptoms that are present for more than about two weeks. There are at least two broad types of a typical depression (Davidson et al. Arch. Gen. Psychiatry, 39, 527-34 (1982); Paykel et al. Psychol. Med., 13,:131-9 (1983); Paykel et al, Arch. Gen. Psychiatry, 39:1041-9 (1982)). One is composed of those depressions accompanied by severe anxiety, and also by phobic symptoms, tension, and pain. The other type of a typical depression is characterized by reversed vegetative symptoms, e.g., increased (rather than decreased) appetite, weight, and sleep. Both of these sets of symptoms are relevant to patients with chronic pain. Many chronic pain patients complain of anxiety (Krishnan et al. Depression as a psychopathological disorder in chronic pain. In: France R. D., Krishnan K. R. R., eds. Chronic pain. Washington, D.C.: American Psychiatric Press, 194-218 (1988); Krishnan et al. Pain, 22:289-94 (1985)) while others complain of weight gain and lethargy. A typical depression is usually considered a nonmelancholic form of depression meaning the neurovegetative symptoms can be reversed. In some recent approaches, for a typical depression to be diagnosed, a typical symptoms must be accompanied by mood reactivity, i.e., “mood regularly improved to at least 50% of normal in response to positive environmental events” (Quitkin et al., Arch. Gen. Psychiatry, 46:787-93 (1989)).

As used herein, depression secondary to pain (DSP) is intended to refer to a depressive disorder characterized by the co-morbidity of pain and a typical depression. Specifically, the pain can be chronic pain, neuropathic pain, or a combination thereof. Specifically, the depression secondary to pain (DSP) can include a typical depression and chronic pain wherein the chronic pain precedes the a typical depression. Alternatively, the depression secondary to pain (DSP) can include a typical depression and chronic pain wherein the a typical depression precedes the chronic pain.

The current method of treatment differs from conventional antidepressant treatment because symptoms of a typical depression do not normally respond to tricyclic antidepressants or selective 5-HT reuptake inhibitors. SNRIs have been shown to treat pain and unexpectedly have been found to be effective in treating both the depression and pain associated with this a typical form of depression.

Chronic pain continues or recurs over a prolonged period of time, caused by various diseases or abnormal conditions, such as rheumatoid arthritis, CFS or FMS. Chronic pain may be less intense that acute pain. The person with chronic pain does not usually display increased pulse and rapid perspiration because the automatic reactions to pain cannot be sustained for long periods of time. Others with chronic pain may withdraw from the environment and concentrate solely on their affliction, totally ignoring their family, their friends, and external stimuli. See, Mosby's Medical, Nursing & Allied Health Dictionary, 5^{th} Edition (1998).

DSP can result from chronic pain in the lower back pain, a typical chest pain, headache, pelvic pain, myofascial face pain, abdominal pain, or neck pain. Alternatively, the chronic pain can be caused by a disease or condition such as arthritis, temporal mandibular joint dysfunction syndrome, traumatic spinal cord injury, multiple sclerosis, CFS, irritable bowel syndrome, chronic fatigue syndrome, premenstrual syndrome, multiple chemical sensitivity, hyperventilation, closed head injury, fibromyalgia, rheumatoid arthritis, diabetes, cancer, HIV, or interstitial cystitis.

Neuropathic pain is usually associated with inflammation or degeneration of the peripheral nerves, cranial nerves, spinal nerves, or a combination thereof. The pain is typically sharp, stinging, or stabbing. The underlying disorder can result in the destruction of peripheral nerve tissue and can be accompanied by changes in the skin color, temperature, and edema. See, Mosby's Medical, Nursing & Allied Health Dictionary, 5^{th} Edition (1998); and Stedman's Medical Dictionary, 25^{th} Edition (1990).

The depression secondary to pain (DSP) can also include a typical depression that includes activity and two or more neurovegetative symptoms such as hypersomnia, increased appetite or weight gain, leaden paralysis, or a long standing pattern of extreme sensitivity to perceived interpersonal rejection; wherein the neurovegetative symptoms are present for more than about two weeks.

An animal model for depression has been developed where an animal develops depression after repeated exposure to a painful stimulus. This experimentally-induced depression called “learned helplessness” resembles the situation when a human develops depression after experiencing chronic uncontrollable pain. There is a large depletion in NE normally observed in this model further supporting the role of the monoamines in depression. In the learned helplessness model, the depression was reversed by infusion of a monoamine oxidase (MAO) inhibitor to remove the depletion of NE (Simson et al Biol Psychiatry 1986 21:724-34). Drugs have been developed which inhibit the degradation and re-uptake of monoamines for the treatment of depression (reviewed in Strolin-Bendetti, Encephale. 1982;8(5):545-85; Rothschild Med Clin North Am 1988 72:765-90; Fuller and Wong Prog Neuropsychopharmacol Biol Psychiatry 1985 9:485-490).

II. Compositions

A. Active Compounds

In one embodiment, DRI compounds, which inhibit the reuptake of serotonin, noradrenaline, are used to prevent or treat individuals with DSP or symptoms of DSP.

Selective norepinephrine (NE)-serotonin (5-HT) reuptake inhibitors (NSRI) are a class of compounds that inhibits the reuptake of both NE and 5-HT where inhibition of reuptake of NE is more than 5-HT. Various techniques are known to determine the norepinephrine (NE)-serotonin (5-HT) reuptake inhibition of a particular NSRI. In one embodiment, the ratio can be calculated from IC₅₀ data for NE and 5-HT reuptake inhibition. For example, it has been reported that for milnacipran the IC₅₀ of norepinephrine reuptake is 100 nM, whereas the IC₅₀ serotonin reuptake inhibition is 200 nM. See, Moret et al., Neuropharmacology, 24(12):1211-1219, 1985; Palmier et al. (1989) Eur J Clin Pharmacol 37(3): 235-8.

The selective NSRI will have an NE: 5-HT reuptake inhibition ratio of at least about 1. Specifically, the selective NSRI can have an NE:5-HT reuptake inhibition ratio of up to about 50. More specifically, the selective NSRI can have an NE: 5-HT reuptake inhibition ratio of about 1:1 to about 50:1. More specifically, the selective NSRI can have an NE: 5-HT reuptake inhibition ratio of about 1:1 to about 20:1. More specifically, the selective NSRI can have an NE: 5-HT reuptake inhibition ratio of about 1:1 to about 5:1. The NSRI should not substantially increase the risk of seizures.

An aminocyclopropane derivative is an aminocyclopropane compound possessing suitable selective norepinephrine (NE)-serotonin (5-HT) reuptake inhibition. Suitable aminocyclopropane derivatives are disclosed, e.g., in U.S. Pat. No. 5,621,142; WO95/22521; Shuto et al., J. Med Chem., 38:2964-2968, 1995; Shuto et al., J. Med. Chem., 39:4844-4852, 1996; Shuto et al., J. Med. Chem., 41:3507-3514, 1998; and Shuto et al., J. Med. Chem., 85:207-213, 2001.

One aminocyclopropane derivative is milnacipran, (±)-cis-2-(aminomethyl)-N,N-diethyl-1-phenylcyclopropanecarboxamide. The CAS Registry Number is 92623-85-3. Methods of preparing milnacipran are disclosed, e.g., in U.S. Pat. No. 4,478,836 and references cited therein. The dextrogyral enantiomer of milnacipran is about twice as active in inhibiting norepinephrine and serotonin reuptake than the racemic mixture, and that the levrogyral enantiomer is much less potent. See, e.g., Viazzo et al., 1996, Tetrahedron Lett. 37(26):4519-4522; Deprez et al., 1998, Eur. J. Drug Metab. Pharmacokinet. 23(2): 166-171). Accordingly, milnacipran can be administered in enantiomerically pure form (e.g., the pure dextrogyral enantiomer) or as a mixture of dextrogyral and levrogyral enantiomers, such as a racemic mixture.

The NE:5-HT of milnacipran is about 2:1. (See, Moret et al. (1985) Neuropharmacology 24(12): 1211-9.) Palmier et al. (1989). Eur J Clin Pharmacol 37(3): 235-8.

Milnacipran and its derivatives also have antagonistic effects at the N-methyl-D-aspartate (NMDA) glutamate receptor. See Shuto et al., 1995, J. Med. Chem., 38:2964-2968; Shuto et al., 1996, J. Med. Chem., 39:4844-4852; Shuto et al., 1998, J. Med. Chem., 41:3507-3514; and Shuto et al., 2001, Jpn. J. Pharmacol., 85:207-213. The SNRI compounds with NMDA receptor antagonistic properties can have IC₅₀ values from about 1 nM-100 μM. For example, milnacipran has been reported to have an IC₅₀ value of about 6.3 μM.

Aminocyclopropane derivatives disclosed in WO95/22521; U.S. Pat. No. 5,621,142; Shuto et al., J. Med. Chem., 38:2964-2968, 1995; Shuto et al., J. Med. Chem., 39:4844-4852, 1996; Shuto et al., J. Med. Chem., 41:3507-3514, 1998; and Shuto et al., Jpn. J. Pharmacol., 85:207-213, 2001 or other compounds that inhibit reuptake of NE more than 5-HT and have NMDA antagonistic properties can be used in treating DSP. Additional information regarding milnacipran may be found in the Merck Index, 12th Edition, at entry 6281.

Another preferred compound is Bicifadine; 1-(4-methyl-phenyl)-3-azabicyclo[3.1.0]-hexane hydrochloride. The CAS Registry number is 71195-57-8. Bicifadine is an NSRI compound with NMDA receptor antagonist properties. Bicifadine has been described as a non-narcotic analgesic compound (Porter et al Curr Therapeutic Res 30; 1981; Wang et al J Clin Pharmacol 1982, 22:160-164).

Another preferred compound is Sibutramine; (cyclobutanemethaneamine or 1(4-chlorophenyl)-N,N-dimethyl-αc-(2-methylpropyl)-, hydrochloride monohydrate). The CAS Registry Numbers are 125494-59-9 [monohydrate], 84485-00-7 [anhydrous]; and 106650-56-0 [sibutramine]. Sibutramine is a TRI compound and blocks the reuptake of the neurotransmitters dopamine, norepinephrine, and serotonin. The chemical structure of sibutramine is well known in the art. This compound is described in U.S. Pat. No. 4,939,175 and Buckett et al.,(Prog. Nuero-Psychopharmacol. & Biol. Psychiat 1988 12:575-584).

Another preferred compound is venlafaxine; (±)-1-1α-[dimethylamino)methyl]-p-methoxybenzyllcyclohexanol hydrochloride. The CAS registry Numbers are 99300-78-4; 93413-69-5. Venlafaxine and synthetic preparations for the same are disclosed, e.g., in U.S. Pat. Nos. 4,535,186; 4,761,501; and references cited therein.

Another preferred compound is duloxetine; 2-thiophenepropanamine, N-methyl-γ-(1-naphthalenyloxy)-hydrochloride. The CAS Registry Number is 116539-59-4. Duloxetine and synthetic preparations for the same are disclosed, e.g., in U.S. Pat. No. 4,956,388; and references cited therein. Tricyclic antidepressants are a well-recognized class of antidepressant compounds and are characterized by a fused tricyclic nucleus. Compounds that are commonly classified as tricyclic antidepressants include imipramine, desipramine, clomipramine, trimipramine, amitriptyline, nortriptyline, doxepin, and protriptyline.

A typical depression is not normally responsive to tricyclic antidepressants and these compounds are not viewed as a first line therapy (Joyce et al N Z J Psychiatry 2002 36:384-391; Stewart et al Neuropsychopharmacology 2002 26:237-245).

B. Salts and Derivatives

Although described above with reference specific to compounds, one can also utilize enantiomers, stereoisomers, metabolites, derivates and salts of the active compounds. Methods for synthesis of these compounds are known to those skilled in the art. Examples of pharmaceutically acceptable salts include, but are not limited to, mineral or organic acid salts of basic residues such as amines, and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Conventional non-toxic salts include those derived from inorganic acids such as hydrochloric, hydrobromic, sulfuric, sulfamic, phosphoric and nitric acid; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, methanesulfonic, ethane disulfonic, oxalic and isethionic acids. The pharmaceutically acceptable salts can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington 's Pharmaceutical Sciences, 17th ed. (Mack Publishing Company, Easton, Pa., 1985, p. 1418) the disclosure of which is hereby incorporated by reference.

A prodrug is a covalently bonded substance which releases the active parent drug in vivo. Prodrugs are prepared by modifying functional groups present in the compound in such a way that the modifications are cleaved, either in routine manipulation or in vivo, to yield the parent compound. Prodrugs include compounds wherein the hydroxy or amino group is bonded to any group that, when the prodrug is administered to a mammalian subject, cleaves to form a free hydroxyl or free amino, respectively. Examples of prodrugs include, but are not limited to, acetate, formate and benzoate derivatives of alcohol and amine functional groups.

A metabolite of the above-mentioned compounds results from biochemical processes by which living cells interact with the active parent drug or other formulas or compounds of the present invention in vivo. Metabolites include products or intermediates from any metabolic pathway.

C. Combinations of Active Ingredients

Selective norepinephrine (NE)-serotonin (5-HT) reuptake inhibitors (e.g., milnacipran) can be administered adjunctively with other active compounds such as antidepressants, analgesics, muscle relaxants, anorectics, stimulants, antiepileptic drugs, and sedative/hypnotics. Specific examples include neurontin, pregabalin, pramipexole, L-DOPA, amphetamine, tizanidine, clonidine, tramadol, morphine, tricyclic antidepressants, codeine, cambamazepine, sibutramine, amphetamine, valium, trazodone and combinations thereof.

The use of an anti-DSP compound disclosed herein can be adjunctively administered with drugs that are known or believed to cause (or precipitate) symptoms of a typical depression. A class of medications that are believed to cause a typical depression are antihypertensives: reserpine, β-blockers such as propranolol, clonidine, methyl-DOPA, and the thiazides. Some of these medications bring about a functional decrease in epinephrine or norepinephrine, neurotransmitters that may be important in the regulation of mood and that may be responsible for the symptoms of depression or the sluggishness that can occur in individuals taking antihypertensives. Additional compounds for treating a typical depression are steroids such as cortisone and prednisone.

D. Formulations and Excipients

The active compounds can be formulated in a pharmaceutical composition comprising a pharmaceutically acceptable carrier and an anti-DSP effective amount of a monoamine reuptake inhibitor. For example, the pharmaceutical composition can comprise a pharmaceutically acceptable carrier and an anti-DSP effective amount of an NSRI such as milnacipran or at least two of milnacipran, sibutramine, and an aminocyclopropane derivative.

The active ingredient can be administered orally in solid dosage forms, such as capsules, tablets and powders, or in liquid dosage forms, such as elixirs, syrups and suspensions. It can also be administered parenterally, in sterile liquid dosage forms. Additives may also be included in the formulation to enhance the physical appearance, improve stability, and aid in disintegration after administration. Liquid dosage forms for oral administration can contain coloring and flavoring to increase patient acceptance. Typical additives include diluters, binders, lubricants, and disintegrants. Gelatin capsules contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours or days. Sustained release products can also be formulated for implantation or transdermal/transmucosal delivery. Such formulations typically will include a polymer that biodegrades or bioerodes thereby releasing a portion of the active ingredient. The formulations may have the form of microcapsules, liposomes, solid monolithic implants, gels, viscous fluids, discs, or adherent films.

Compressed tablets can be sugar coated or film coated to mask any unpleasant taste and protect the tablet from the atmosphere, or enteric coated for selective disintegration in the gastrointestinal tract. They can also be formulated to release the drug substance in a manner to provide medication over a period of time. There are a number of types which include delayed-action tablets in which the release of the drug substance is prevented for an interval of time after administration of until certain physiological conditions exist; repeat-action tablets which periodically release a complete dose of the drug substance to the gastrointestinal fluids; and the extended-release tablets which continuously release increments of the contained drug substance to the gastrointestinal fluids.

Compressed tablets may be characterized or described by a number of specifications. These include the diameter size, shape, thickness, weight, hardness, and disintegration time. Film-coated tablets are compressed tablets, which are covered with as thin layer of film or water-soluble material. A number of polymeric substances with film-forming properties may be used. Film coating imparts the same general characteristics as sugar coating with the added advantage of a greatly reduced time period required for the coating operation.

Enteric-coated tablets are compressed tablets coated with substances that resist solution in gastric fluid but disintegrate in the intestine. Enteric coatings can be used for tablets containing drug substances which are inactivated or destroyed in the stomach, for those which irritate the mucosa, or as a means of delayed release of the medication.

Multiple compressed tablets are compressed tablets made by more than one compression cycle. Layered tablets are prepared by compressing additional tablet granulation on a previously compressed granulation. The operation my be repeated to produce multilayered tablets of two or three layers. Special tablet presses are required to make layered tablets.

Press-coated tablets, which are also referred to as dry-coated, are prepared by feeding previously compressed tablets into a special tableting machine and compressing another granulation layer around the preformed tablets. They have all the advantages of compressed tablets, i.e., slotting, monogramming, speed of disintegration, etc., while retaining the attributes of sugar-coated tablets in masking the taste of the drug substance in the core tablets. Press-coated tablets can also be used to separate incompatible drug substances; in addition, they can provide a means to give an enteric coating to the core tablets. Both types of multiple-compressed tablets have been widely used in the design of prolonged-action dosage forms.

Oral agent, oral compound are compounds that may be orally administered. Although it is preferable that component (a) and component (b) both be administered by the same route (that is, for example, both orally) or dosage form, if desired, they may each be administered by different routes (that is, for example, one component of the combination product may be administered orally, and another component may be administered intravenously) or dosage forms.

The combination products may be formulated such that, although the active ingredients are combined in a single dosage unit, the physical contact between the active ingredients is minimized. In order to minimize contact, for example, where the product is orally administered, one active ingredient may be enteric coated. By enteric coating one of the active ingredients, it is possible not only to minimize the contact between the combined active ingredients, but also, it is possible to control the release of one of these components in the gastrointestinal tract such that one of these components is not released in the stomach but rather is released in the intestines.

In another embodiment where oral administration is desired, one active ingredient is coated with a sustained-release material which effects a sustained-release throughout the gastrointestinal tract and also serves to minimize physical contact between the combined active ingredients. The sustained-released component can be additionally enteric coated such that the release of this component occurs only in the intestine. Still another approach involves the formulation of a combination product in which the one component is coated with a sustained and/or enteric release polymer, and the other component is also coated with a polymer such as a low-viscosity grade of hydroxypropyl methylcellulose or other appropriate materials as known in the art, in order to further separate the active components. The polymer coating serves to form an additional barrier to interaction with the other component. In each formulation wherein contact is prevented between components (a) and (b) via a coating or some other material, contact may also be prevented between the individual agents of component (b).

Dosage forms wherein one active ingredient is enteric coated can be in the form of tablets such that the enteric coated component and the other active ingredient are blended together and then compressed into a tablet or such that the enteric coated component is compressed into one tablet layer and the other active ingredient is compressed into an additional layer. Optionally, in order to further separate the two layers, one or more placebo layers may be present such that the placebo layer is between the layers of active ingredients. In addition, dosage forms can be in the form of capsules wherein one active ingredient is compressed into a tablet or in the form of a plurality of microtablets, particles, granules or non-perils, which are then enteric coated. These enteric coated microtablets, particles, granules or non-perils are then placed into a capsule or compressed into a capsule along with a granulation of the other active ingredient. These as well as other ways of minimizing contact between the components of combination products, whether administered in a single dosage form or administered in separate forms but at the same time or concurrently by the same manner, will be readily apparent to those skilled in the art, based on the present disclosure.

Pharmaceutical kits useful for the treatment of DSP, related diseases and symptoms, include a therapeutically effective amount of a pharmaceutical composition that includes a compound of component (a) and one or more compounds of component (b), in one or more sterile containers. Component (a) and component (b) may be in the same sterile container or in separate sterile containers. The sterile containers of materials may comprise separate containers, or one or more multi-part containers, as desired. Component (a) and component (b), may be separate, or physically combined into a single dosage form or unit as described above. Such kits may further include, if desired, one or more of various conventional pharmaceutical kit components, such as for example, one or more pharmaceutically acceptable carriers, additional vials for mixing the components, etc., as will be readily apparent to those skilled in the art. Instructions, either as inserts or as labels, indicating quantities of the components to be administered, guidelines for administration, and/or guidelines for mixing the components, may also be included in the kit.

Typically, water, suitable oil, saline, aqueous dextrose (glucose), and related sugar solutions and glycols such as propylene glycol or polyethylene glycols are suitable carriers for parenteral solutions. Solutions for parenteral administration preferably contain a water-soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts, and sodium EDTA. In addition, parenteral solutions can contain preservatives, such as benzalkonium chloride, methyl- or propyl-paraben and chlorobutanol. Suitable pharmaceutical carriers are described in Remington's Pharmaceutical Sciences, supra, a standard reference text in this field.

III. Methods of Use

A. Patients/Individuals to be Treated

For therapeutic use, the monoamine reuptake inhibitor compound typically will be administered to an individual expressing symptoms of pain or DSP. For prophylactic treatment, the monoamine reuptake inhibitor compound typically will be administered to a patient expressing symptoms of chronic or neuropathic pain even though a diagnosis of DSP may not have yet been made. Alternatively, prophylactic administration may be used to avoid the onset of the symptoms of the underlying disorder, particularly if the symptom manifests cyclically. In this latter embodiment, the therapy is prophylactic with respect to the associated physiological symptoms instead of the underlying indication. For example, the compound could be prophylactically administered prior to bedtime to avoid the sleep disturbances associated with DSP. Alternatively, the compound could be administered prior to recurrence or onset of a particular symptom (for example, pain, or fatigue).

Conventional antidepressant medications such as tricyclic antidepressants and selective 5-HT reuptake inhibitors are ineffective in treating symptoms of a typical depression such as anxiety and pain. Antidepressant medications with analgesic properties are candidates for use in this method to treat the a typical depression as well as the pain component associated with it. The pain component of DSP, which can be chronic pain or neuropathic pain, can be treated at the same time as the a typical depressive component characteristic of DSP.

B. Effective Dosage Ranges

The administered dosage will vary depending upon known factors, such as the pharmacodynamic characteristics of the particular agent and its mode and route of administration; the age, health and weight of the recipient; the nature and extent of the symptoms; the kind of concurrent treatment; the frequency of treatment; and the effect desired. A daily dosage of active ingredient can be expected to be about 0.001 to about 1000 milligrams per kilogram of body weight, with the preferred dose being about 0.1 to about 100 mg/kg, preferably administered several times a day.

Dosage forms of compositions suitable for administration contain from about 20 mg to about 500 mg of active ingredient per unit. Preferably the compound is administered in about 100 mg/day to about 250 mg/day. In another embodiment, the compound can be administered up to about 400 mg/day. In these pharmaceutical compositions the active ingredient will ordinarily be present in an amount of about 0.5-95% by weight based on the total weight of the composition.

The dosage will preferably be an effective amount to alleviate symptoms of DSP such as pain, anxiety, and neurovegetative symptoms such as fatigue, hypersomnia and hyperphagia.

C. Methods of Administration

The compounds can be administered to treat pain-associated depression (PAD), and related diseases and symptoms, by any means that produces contact of the active agent with the agent's site of action in the body of a mammal. They can be administered by any conventional means available for use in conjunction with pharmaceuticals, either as individual therapeutic agents or in a combination of therapeutic agents. They can be administered alone, but preferably are administered with a pharmaceutical carrier selected on the basis of the chosen route of administration and standard pharmaceutical practice.

The active compound is preferably administered one or more (e.g., 1, 2, 3, 4, or 5) times per day.

Such compositions can be administered orally, buccally, parenterally, by inhalation spray, rectally, intradermally, transdermally, or topically in dosage unit formulations containing conventional nontoxic pharmaceutically acceptable carriers, adjuvants, and vehicles as desired. The term parenteral as used herein includes subcutaneous, intravenous, intramuscular, or intrasternal injection, or infusion techniques. In the preferred embodiment the composition is administered orally.

Any patent, patent document, or reference disclosed herein is incorporated by reference. The present invention will be further understood by reference to the following non-limiting examples.

### EXAMPLE 1

### Therapeutic Effect of Milnacipran on Swim Stress-Induced Muscle Hyperalgesia

Repeated inescapable swim stress produces a delayed and long-term cutaneous hyperalgesia to both brief thermal and prolonged chemical stimuli in rats. The swim stress-induced hyperalgesia (SSIH) model displays cutaneous hyperalgesia involving NMDA receptor mechanisms. The SSIH rat model provides a useful animal model of “learned helplessness” where a repeated inescapable stressful event ultimately leads to depression. Chronic pain states develop in these rats resulting from the repeated stressful event.

Methods

Sprague-Dawley rats weighing (200-300 g) were used in these experiments. Dosage groups consisted of milnacipran in 1, 10 and 30 mg/kg doses as well as uninjected and saline-only controls. Milnacipran was obtained from Pierre-Fabre (France), mixed in normal saline and administered via intraperitoneal injection (i.p.). The stress was applied for 10 mins/day and consisted of sham swim test in 2-3 cm of water (where the animal's feet touched the bottom of the tank), forced swim test in 20 cm of water or no handling at all (i.e. animal left in its cage). Animals were tested before and after the stress to establish baseline and post-stress responses. The parameters were designed to measure muscle hyperalgesia by measuring grip strength (in kgs by algometer) as well as thermal nociception threshold by latency response to hot plate stimulus (in seconds).

Results

Treatment with milnacipran had no effect on preventing the decreased hot plate latency response after forced swim stress. Thermal cutaneous hyperalgesia evoked by swim stress is persistent and remains essentially unchanged for several days post conditioning for all milnacipran doses tested as well as saline-only and uninjected controls.

Muscle hyperalgesia was tested by measuring grip strength before and after stress. Swim stress followed by repeated IP injection reduces grip strength that appears to be associated with a muscular allodynia. Saline-only groups displayed decreased grip strength after forced swim stress. Milnacipran reverses the decreased grip strength evoked by swim stress at all doses tested.

### CONCLUSIONS

Thermal cutaneous hyperalgesia is not responsive to milnacipran (1-50 mg/kg) and persists unchanged for several days after the stressful event. Muscle hyperalgesia responds well to milnacipran (1-50 mg/kg) reversing the decrease in grip strength seen after forced swim stress. Modulation of cutaneous and muscular nociception can be dissociated in this animal model since they can exist and be pharmacologically affected independently. The SSIH model demonstrates that milnacipran can be administered to prevent the onset of chronic intractable hyperalgesia developing after inescapable swim stress and can be a potential candidate to treat or prevent pain-associated depression.

### EXAMPLE 2

### Treatment of Depression and Pain in Human Subjects with Once or Twice Daily Milnacipran Treatment.

Methods:

Patients suffering from depression were treated in a double-blind, placebo-controlled trial. Subjects were divided into three test groups: milnacipran dose once a day; milnacipran dose twice a day; and placebo group. Subjects completing the 12 week trial for each test group consisted of: milnacipran dose once a day (n=32); milnacipran dose twice a day (n=37); and placebo group (n=21).

Pain in the test subjects was detected by electronic diary where once a day they were asked to rate their pain on a 20 point scale. Pain was rated for two weeks before treatment to establish a baseline and then for two weeks at the end of the study. Pain was rated also by random prompting where subjects were randomly called 4-5 times/day to rate their pain on the 20 point scale. Only responders were included in the analysis where a responder was defined as having a 4 point reduction on the scale at the endpoint of the study.

Subjects were also rated on the Beck Depression Inventory at the beginning and end of the study. The BDI is a self-administered 21 item self-report scale measuring manifestations of depression. Only subjects completing the 12 week study were included in the analysis.

Subjects were also rated by patient global score at the end of the study where they were asked to rate their general condition on a seven point scale in comparison to their condition at the beginning of the study. (1-3 worse; 4 no change; 5-7 better) Data was compiled and analyzed for observed cases (OC) who completed the 12 weeks study.

Results:

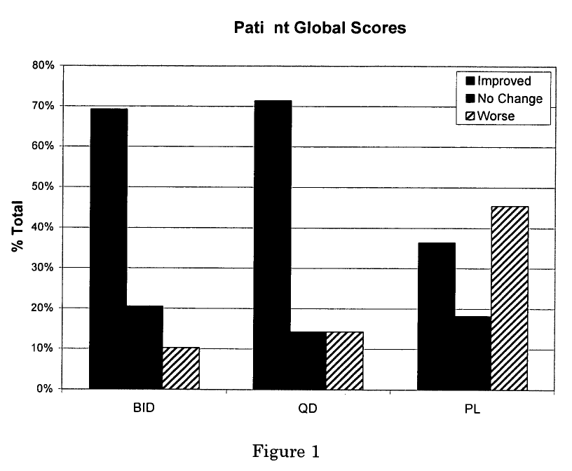

Patient Global Scores in both once-a-day and twice-a-day milnacipran treatment groups were significantly improved over placebo group (FIG. 1). For both milnacipran groups, 70% of the subjects reported global improvement in their condition over only 35% in the placebo group. The results were very similar despite the difference in daily dosing regimen. Only 10% of the milnacipran-treated subjects reported a worsening in their condition as compared to 45% of the placebo group.

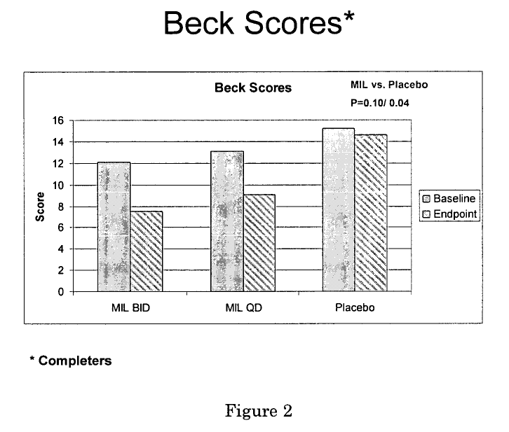

The results of the Beck Depression Index indicated that treatment with milnacipran ameliorated depression regardless of whether a once-a-day or twice-a-day dose was administered (FIG. 2). Test subjects in both groups demonstrated a 35-40% reduction in Beck score after treatment with Milnacipran. Changes in Beck score were comparable indicating that a once-a-day or twice-a-day dose of milnacipran was similarly effective in treating depression. There was no significant change in Beck score of the placebo group at the end of the study.

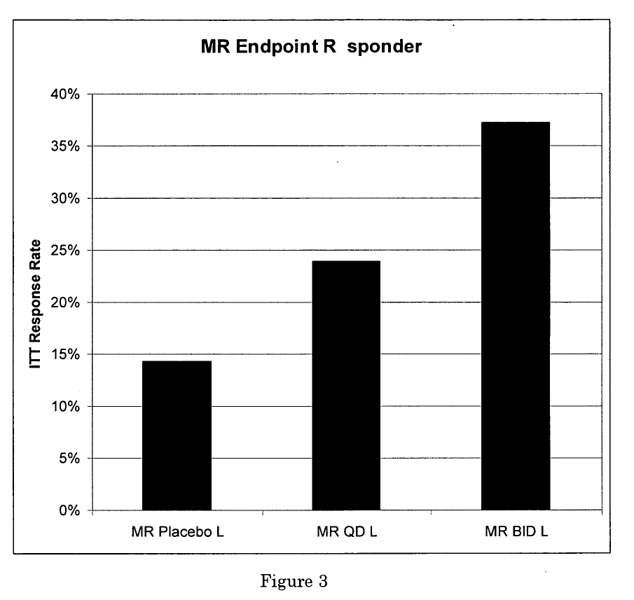

Milnacipran ameliorated pain in the test subjects as demonstrated in a binary responder analysis (FIG. 3). The once-a-day dose of milnacipran improved ITT Response Rate over the placebo group while the twice-a-day dose significantly improved the ITT Response Rate over both the once-a-day dose and the placebo group. Unexpectedly, despite the similar effects of the milnacipran dosing regime on patient global scores and Beck scores, a similar relationship was not observed in treating pain the subjects.

### CONCLUSIONS

These results demonstrate the efficacy of milnacipran in treating both depression and pain. The similarity in patient global scores and Beck scores regardless of whether milnacipran was administered once or twice a day was surprising because of the difference in dosing regimen. Presumably milnacipran which is rapidly absorbed and possesses a half life of approximately 8 hours would be cleared out of the body with only a once-a-day administration. These results indicate that a constant circulating dose of milnacipran may not be necessary to treat depression and improve a patients condition.

Another unexpected finding of this study was the different effects of milnacipran dose on the subjects experience of pain. The test subjects reported an improved condition with a once-a-day dose of milnacipran and an even greater improvement with a twice-a-day dose. This is contrary to the virtually identical effects of the milnacipran dosing regimens on depression and patient global scores. These results suggest a dissociation of the actions of milnacipran on treating the pain and depression components.

Those skilled in the art will recognize, or be able to ascertain using no more than routine experimentation, many equivalents to the specific embodiments of the invention described herein. Such equivalents are intended to be encompassed by the following claims.

We claim:

1. A method of treating or preventing a typical depression secondary to pain (DSP) in an individual suffering from DSP or at risk thereof, the method comprising administering to the individual an effective amount of a dual norepinephrine serotonin reuptake inhibitor (NSRI) or triple reuptake inhibitor (TRI) to alleviate or prevent at least one symptom of a typical depression.

2. The method of claim 1 wherein the selective NSRI has an NE: 5-HT reuptake inhibition ratio of about 1:1 to about 50:1.

3. The method of claim 1 wherein the selective NSRI has an NE: 5-HT reuptake inhibition ratio of about 1:1 to about 20:1.

4. The method of claim 1 wherein the selective norepinephrine (NE)-serotonin (5-HT) reuptake inhibitor (NSRI) is milnacipran: 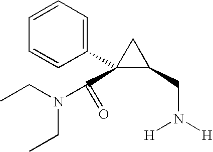
or sterioisomeric forms, mixtures of sterioisomeric forms, metabolites, derivatives, or pharmaceutically acceptable salts thereof.

5. The method of claim 4 wherein the milnacipran is administered at a dosage of between 100 and 400 mg/day.

6. The method of claim 4 wherein the milnacipran is administered at a dosage of between 100 and 250 mg/day.

7. The method of claim 1 wherein the inhibitor is administered two or more times per day.

8. The method of claim 1 wherein the NSRI has NMDA receptor antagonist properties.

9. The method of claim 1 wherein the selective norepinephrine (NE)-serotonin (5-HT) reuptake inhibitor (NSRI) does not substantially increase the risk of seizures.

10. The method of claim 1 wherein the selective norepinephrine (NE)-serotonin (5-HT) reuptake inhibitor (NSRI) comprises at least two of milnacipran, sibutramine, and an aminocyclopropane derivative.

11. The method of claim 1 wherein the inhibitor is a triple reuptake inhibitor blocking uptake of serotonin, noradrenaline, and dopamine.

12. The method of claim 1 wherein the DSP comprises a typical depression and either chronic pain or neuropathic pain.

13. The method of claim 12 wherein the DSP comprises chronic pain selected from the group consisting of lower back pain, a typical chest pain, headache, pelvic pain, myofascial face pain, abdominal pain, neck pain and chronic pain caused by a disease or condition.

14. The method of claim 1 wherein the DSP comprises a typical depression characterized by mood reactivity and neurovegetative symptoms present for more than about two weeks.

15. A pharmaceutical composition comprising a pharmaceutically acceptable carrier and an effective anti-DSP amount of a dual serotonin-norepinephrine reuptake inhibitor or triple reuptake inhibitor to alleviate or prevent one or more of the symptoms characteristic of a typical depression.

16. The pharmaceutical composition of claim 15 comprising milnacipran in a formulation delivering between 100 and 400 mg/day.

17. The pharmaceutical composition of claim 15 comprising a reuptake inhibitor in a dosage greater than the dosage required to treat typical depression or pain.

18. The pharmaceutical composition of claim 15 comprising at least two of milnacipran, sibutramine, and an aminocyclopropane derivative.
